(19) United States
(12) Patent Application Publication
(10) Pub. No.: US 2005/0267449 A1
(43) Pub. Date: Dec. 1, 2005

(54) Surgical instrument positioning system

(75) Inventors: John K. Edoga, North Beach, NJ (US); Thierry Richard, Florham Park, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Edrich Health Technologies, Inc., Morristown, NJ (US)

(21) Appl. No.: 11/133,015

(22) Filed: May 19, 2005

Related U.S. Application Data
(63) Continuation-in-part of application No. 10/690,291, filed on Oct. 21, 2003.
(60) Provisional application No. 60/419,949, filed on Oct. 21, 2002.
(60) Provisional application No. 60/452,148, filed on Mar. 5, 2003.
(60) Provisional application No. 60/476,497, filed on Jun. 6, 2003.
(60) Provisional application No. 60/572,660, filed on May 19, 2004.
(60) Provisional application No. 60/577,029, filed on Jun. 4, 2004.

(51) Int. Cl. A61B 17/00

(52) U.S. Cl. 606/1

(57) ABSTRACT

An assembly for supporting a surgical platform, the assembly comprising an attachment mechanism for securement to a surgical table, a post extending from the attachment mechanism at its first end, a platform mating device engaged with the post's second end at a base portion, the platform mating device comprising at least one leg extending from the base portion, the at least one leg adapted to be mated with a surgical platform to support the surgical platform. Also disclosed is a post for mounting a surgical apparatus to a surface, the post comprising a first end and a second end, the first end operatively engaged with the surface and the second end operatively engaged with the apparatus, a middle portion between the first end and the second end, the middle position having at least one condition in which the middle portion is non-linear.

### CROSS REFERENCE TO RELATED APPLICATIONS

The present application is a continuation-in-part of U.S. patent application Ser. No. 10/690,291 filed Oct. 21, 2003, which claims the benefit of U.S. Provisional Patent Application Ser. No. 60/419,949 filed Oct. 21, 2002, U.S. Provisional Application Ser. No. 60/452,148 filed Mar. 5, 2003, and U.S. Provisional Application Ser. No. 60/476,497 filed Jun. 6, 2003. This application also claims the benefit of the filing date of U.S. Provisional Patent Application No. 60/572,660 filed May 19, 2004 and 60/577,029 filed Jun. 4, 2004. The disclosures of each of the foregoing applications are hereby incorporated herein by reference.

### BACKGROUND OF THE INVENTION

In addition to the aforementioned relationships, the present invention is directed toward improvements in systems of the type described in U.S. Pat. No. 5,728,047 issued to Edoga, one of the present inventors, the disclosure of which is incorporated herein by reference. In U.S. Pat. No. 5,728,047, there is provided a surgical instrument positioning system including a belt assembly positionable about the body of a patient which may be utilized during surgical procedures to assist with tasks previously performed by surgeons. Many of these tasks were considered “robotic,” and require only a minimal amount of skill and adaptation.

For example, laparoscopic cholecystectomy, i.e. the removal of the gallbladder, is typically performed by at least two surgeons. An assistant surgeon provides retraction of the right lobe of the liver by grasping the upper portion of the gallbladder using a ratcheted grasper and pushes the liver forward and to the right. Depending on the operating surgeon's preference, the assistant surgeon either also provides additional traction on the gallbladder by grasping the portion of the gallbladder which leads to the bile ducts or he or she holds the laparoscope. The need to use at least two surgeons makes this procedure unnecessarily costly.

Laparoscopic Nissen fundoplication, i.e., the repair of hiatal hernias and the creation of a valve to check acid reflux from the stomach into the esophagus, is currently performed by at least three surgeons, increasing costs even more. The first assistant surgeon usually holds the laparoscope and also provides the needed traction on the stomach and esophagus while the second assistant surgeon retracts the lateral segment of the left lobe of the liver, in most cases, using a fan retractor.

Besides the increase in costs associated with the need for additional surgeons to mechanically hold surgical instruments in place, problems can also arise during the surgical procedure due to a number of human factors. These problems may include the surgeon becoming fatigued or distracted while trying to hold the instrument in a fixed position, or the surgeon unintentionally permitting the instrument to drift from its original position due to a lack of visual reinforcement of the instrument's position within the patient as the surgeon holding the retractor rarely gets a chance to see the retractor position, especially during the critical stages of the procedures. Moreover, the presence of additional surgeons about the patient can interfere with the operative field and the arm movements of the lead surgeon.

There have been a number of attempts to provide positioning systems for holding retractors and other surgical instruments in a fixed position so as to eliminate the need for human involvement. One example provides for a “stepped” surgical retractor including a ladder-like support which is attached via a universal clamp to the operating table and projects vertically upwardly with respect to the operating table. Although devices of this type eliminate the need for a surgeon to hold the retractor in a fixed position, the support structure of the device can interfere with the arms of the surgeon due to its vertical projection into the operative field. It may also press against the body of a wide or obese patient and may lead to pressure ulcers of the skin.

Other retractor or positioning systems provide mechanical arm devices which are clamped to the side of the operating table and employ a pivoting robotic arm which extends horizontally over the table and patient. These systems also suffer from problems in that they are located in the operative area above the patient and can interfere with the movements of the surgeon. These devices can also be cumbersome and difficult to use. Furthermore, such systems are relatively expensive, especially when provided with hydraulic or motor-assisted lifting mechanisms.

The surgical instrument positioning system developed by the present inventors and disclosed in U.S. Pat. No. 5,728,047 and related applications provided great advancements over the prior art. For example, it provided a positioning system for use during surgical procedures, such as during laparoscopic surgery, which was relatively simple to use, inexpensive, and which solved the problems associated with the use of assistant surgeons and positioning systems which interfered with the surgeon. However, it has been discovered that under certain conditions, the positioning system may be difficult to use. For example, in the case of obese patients, the positioning support tray may be difficult to position in a fixed relation relative to the patient. Rather, the support tray may rotate about the longitudinal axis of the patient as it is used and becomes loosened.

Accordingly, a positioning system of the type disclosed in U.S. Pat. No. 5,728,047 and related applications would provide additional benefits if affixed directly to the surgical table, rather than to the patient, to avoid any patient-related deficiencies.

### SUMMARY OF THE INVENTION

The aforementioned shortcomings of the prior instrument positioning systems have been addressed by the present invention, which in one embodiment comprises an assembly for supporting a surgical platform, the assembly comprising an attachment mechanism for securing the assembly to a surgical table, a post extending from the attachment mechanism, the post having a first end at the attachment mechanism and a distal second end, a platform mating device engaged with the second end of the post at a base portion, the platform mating device comprising at least one leg extending from the base portion, the at least one leg adapted to be mated with a surgical platform to support the surgical platform.

The at least one leg may further comprise a notch adapted to be mated with a locking member of a surgical platform, wherein the locking member of the surgical platform may enter the notch to restrict relative movement between the surgical platform and the at least one leg.

The at least one leg may comprise two legs, at least one of the legs having a notch adapted to be mated with a locking member of a surgical platform, wherein the locking member of the surgical platform may enter the notch to restrict relative movement between the surgical platform and the two legs.

The first end of the post may be relatively straight and may be adapted to be adjustably mated with the attachment mechanism.

The post may comprise a series of links arranged in tandem.

The post may further comprise an internal wire, the wire serving to at least partially support the post.

The links may include aligned apertures therethrough, the wire nearly filling the entire apertures.

The wire may be malleable such that movement of the post by a user bends the wire into a fixed position at least partially supporting the post.

The post may further comprise an internal wire, the wire having a loose first condition and a taught second condition, the post being malleable when the wire is in the first condition and fixed when the wire is in the second condition.

The post may further comprise a post tensioning mechanism, the post tensioning mechanism adapted to selectively tighten or looses the wire within the post.

The post may be curved.

In accordance with another aspect of the present invention, a post for mounting a surgical apparatus to a surface may comprise a first end and a second end, the first end operatively engaged with the surface and the second end operatively engaged with the apparatus, a middle portion between the first end and the second end, the middle portion having at least one condition in which the middle portion is non-linear.

The post may comprise a series of links arranged in tandem.

Each of the links may include aligned apertures, the post further comprising a wire running along the length of the post within the apertures.

The wire may leave an annular space between the wire and the links.

The wire may be malleable such that movement of the post by a user bends the wire into a fixed position at least partially supporting the post.

The wire may at least partially support the post.

The wire may have a first condition in which the wire is loose and a second condition in which the wire is tight, the post being flexible in the first condition and rigid in the second condition.

The post may further comprise a post tensioning device operative to selectively tighten or loosen the wire within the post.

The links may comprise features on their end surfaces to increase friction between each of the links.

The links may include grooves on their end surfaces.

In accordance with a further aspect of the present invention, an assembly for supporting a surgical platform may comprise an attachment mechanism for securing the assembly to a surface, a post extending from the attachment mechanism, a platform mating mechanism engaged with the post at a base portion opposite the attachment mechanism, the platform mating mechanism comprising a pair of opposed supports operative to selectively move toward each other or away from each other, the supports adapted to be mated with a surgical platform to support the surgical platform.

The platform mating mechanism may further comprise a base having a channel therein, the supports moveable within the channel.

The supports may include tapered outside edges.

In accordance with a still further aspect of the present invention, a surgical platform system may comprise a surgical platform, the surgical platform comprising an underside having a recessed channel therein, a platform support mechanism, the platform support mechanism comprising an attachment mechanism for attachment of the platform support mechanism to a surface, a platform mating mechanism, and a post extending between the attachment mechanism and the platform mating mechanism, the platform mating mechanism comprising a pair of adjustable supports, the supports adapted to be placed within the channel.

The supports may have a first condition in which the supports are spaced apart from each other and a second condition in which the supports are moved together, the supports adapted to be matingly engaged with the recessed channel when the supports are in the first condition.

The mating engagement may be by way of frictional forces between the supports and the recessed channel.

The supports may be tapered.

The recessed channel may be correspondingly tapered such that the tapered supports and the tapered recessed channel affix the surgical platform to the platform by more than friction alone.

In accordance with an additional aspect of the present invention, a surgical platform may comprise a main portion and at least one shaped passage below the main portion, the shaped passage adapted to permit entry of a platform mating device for supporting the platform.

The at least one shaped passage may comprise a rectangular cross-section.

The surgical platform may further comprise a locking device associated with the shaped passage, the locking device having a first position in which the locking device at least partially enters the shaped passage and a second position in which the locking device is not within the shaped passage.

The shaped passage may include a longitudinal centerline and the locking device may enter the shaped passage normal to the longitudinal centerline.

In accordance with yet another aspect of the present invention, a device for attaching a surgical accessory to a surgical platform having a handle and a main portion may comprise a jaw mechanism, the jaw mechanism comprising a first jaw adapted to fit between the handle of the surgical platform and the main section of the surgical platform and a second jaw adapted to be outside the handle such that the jaw may be closed to squeeze the handle of the surgical platform to support the device.

The device may further comprise a post, the post adapted to support a surgical accessory.

The device may further comprise a jaw manipulation component, wherein the jaw manipulation component may be adapted to move the jaw from an open position to a closed position to selectively squeeze the handle.

The first jaw and the second jaw may be curved.

The first jaw and the second jaw may have a geometry corresponding to the shape of the handle.

### BRIEF DESCRIPTION OF THE DRAWINGS

The subject matter regarded as the invention is particularly pointed out and distinctly claimed in the concluding portion of the specification. The invention, however, both as to organization and method of operation, together with features, objects, and advantages thereof will be or become apparent to one with skill in the art upon reference to the following detailed description when read with the accompanying drawings. It is intended that any additional organizations, methods of operation, features, objects or advantages ascertained by one skilled in the art be included within this description, be within the scope of the present invention, and be protected by the accompanying claims.

In regard to the drawings, FIG. 1 perspective view of a surgical instrument support tray in accordance with certain aspects of the present invention;

FIG. 2 is a perspective view of a support device in accordance other aspects of the present invention;

FIG. 3 depicts a blown-up view of portions of the support device of FIG. 2;

FIG. 4 is a cross-sectional view of the instrument support tray of FIG. 1;

FIG. 5 is a cross-sectional view of the instrument support tray of FIG. 1;

FIG. 6 is a perspective view of a support device in accordance with further aspects of the present invention;

FIG. 7A is a cross-sectional view of a portion of the support device of FIG. 6 in accordance with one embodiment of the present invention;

FIG. 7B is a cross-sectional view of a portion of the support device of FIG. 6 in accordance with a further embodiment of the present invention;

FIG. 8 is a bottom plan view of a surgical instrument support tray in accordance with other aspects of the present invention;

FIG. 9 is a perspective view of a surgical instrument support tray in proximity to a mounting mechanism;

FIG. 10 is a perspective view of a surgical instrument support tray in proximity to an accessory; and,

FIG. 11 is a perspective view of an accessory being mounted upon a surgical instrument support tray.

### DETAILED DESCRIPTION

In the following is described the embodiments of the surgical instrument positioning system and method of use of the present invention. In describing the embodiments illustrated in the drawings, specific terminology will be used for the sake of clarity. However, the invention is not intended to be limited to the specific terms so selected, and it is to be understood that each specific term includes all technical equivalents that operate in a similar manner to accomplish a similar purpose.

FIG. 1 depicts an exemplary surgical platform 100 in accordance with certain aspects of the present invention. Surgical platform 100 is preferably formed from a single piece of material, such as plastic, which may be rigid or flexible. Surgical platform 100 may comprise a first side edge 102 and second side edge 104. Between the two side edges, may be a cephalad edge 106 and a caudad edge 108. The four edges, 102, 104, 106, 108, combine to form a generally rectangular and flat surgical platform main portion 101.

As discussed in U.S. patent application Ser. No. 10/690,291, and other related applications, accessories may be utilized with the surgical platform 100 to assist the surgical team with the required tasks. Such accessories include anti-rotational retractor retention systems, simple retractors, or the like. The surgical platform 100 may include features for attachment of such accessories, for example hooks 110, loops 112, or apertures 114. The use of such hooks 110, loops 112, apertures 114, as well as additional features are shown and described in U.S. patent application Ser. No. 10/690,291, incorporated herein by reference.

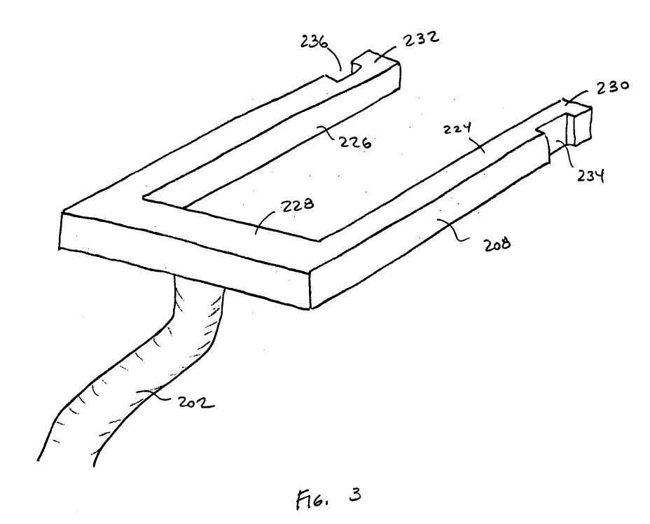

In addition to the features shown and described in relation to U.S. patent application Ser. No. 10/690,291, the surgical platform 100 may include features unique to the present invention. Such features may include shaped passages, such as the first shaped passage 116 shown in FIG. 1 (FIG. 4 shows a second shaped passage 118 in addition to the first shaped passage). As will be discussed, the shaped passages 116, 118 are adapted to enable the surgical platform 100 to become engaged with a support device.

Additional features of the surgical platform may include handles 120, 122, located at each of the sides edges 102, 104. The first side edge handle 120 and the second side edge handle 122, as will be discussed, may be utilized to carry the surgical instrument platform 100, in the conventional manner when the surgical instrument platform is unattached from its support, but may also be utilized as support surfaces for various components.

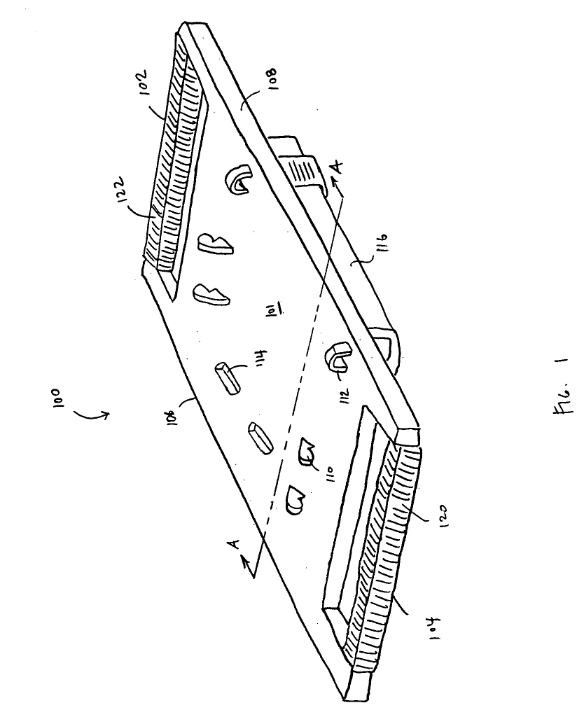

Moving to FIG. 2, there is depicted a perspective view of a support device 200, which may be utilized to support the surgical platform 100. The support device 200 may comprise a post 202 cooperating with a mounting mechanism 204 to attach the post to a rail 206 provided on the side of a conventional surgical table (not shown). The mounting mechanism 204 may be adapted to mate with existing rails 206, or the rail may be customized for the mounting mechanism.

The post 202 preferably attaches to a platform mating device 208 at a first end 210 spaced away from the post's second end 212, located at the mounting mechanism 204.

The mounting mechanism 204 may include an aperture 214 formed therein for insertion of the second end 212 of the post 202. The second end 212 of the post 202 is preferably straight, such that the second end may freely slide within the aperture 214 when so desired.

The mounting mechanism 204 may be adapted to engage the rail 206 provided on a surgical table (not shown). The mounting mechanism 204 may be provided with a handle actuator 216 adapted to selectively unlock the mounting mechanism such that the mounting mechanism may slide along the rail 206 or lock the mounting mechanism such that the mounting mechanism may not freely slide along the rail. The handle actuator 216 may also lock the second end 212 of the post 202 therein, to selectively position the post. In this regard, it will be appreciated that the post 202 may be inserted to various levels through the aperture 212 to raise or lower the level of the platform mating device 208 at the first end 210 of the post. Lowering will preferably bring the platform mating device 208 closer to the patient (not shown) laying upon the surgical table (not shown) while raising will bring the platform farther away. The post 202 may include a collar 218 or other feature to prevent the post from being inserted beyond a predetermined level. In some embodiments, the collar 218 may be adjustable, such as by use of a set screw 220, such that the height of the collar may be adjusted and fixed at a desired level.

Between the first end 210 and the second end 212 of the post 202, a middle portion 222 of the post may be curved to accommodate a large or obese patient, a patient that may otherwise have appendages extending over the edge of the surgical table, or to accommodate other apparatus within the surgical arena. The curved middle portion 222 of the post 202 may be an exaggerated curve, such as shown in FIG. 1, or may only be slightly bent, as required for the particular application.

In other embodiments, the middle portion 222 of the post 202 may be straight. In this regard, the post 202 may comprise a straight cylinder extending from the first end 210 to the second end 212 of the post.

Whether the middle portion 222 of the post 202 is straight or curved, the second end 212 is preferably straight such that the second end may freely slide within the aperture 214 of the mounting mechanism 204 to slide the post up and down, thus raising or lowering the platform mating device 208.

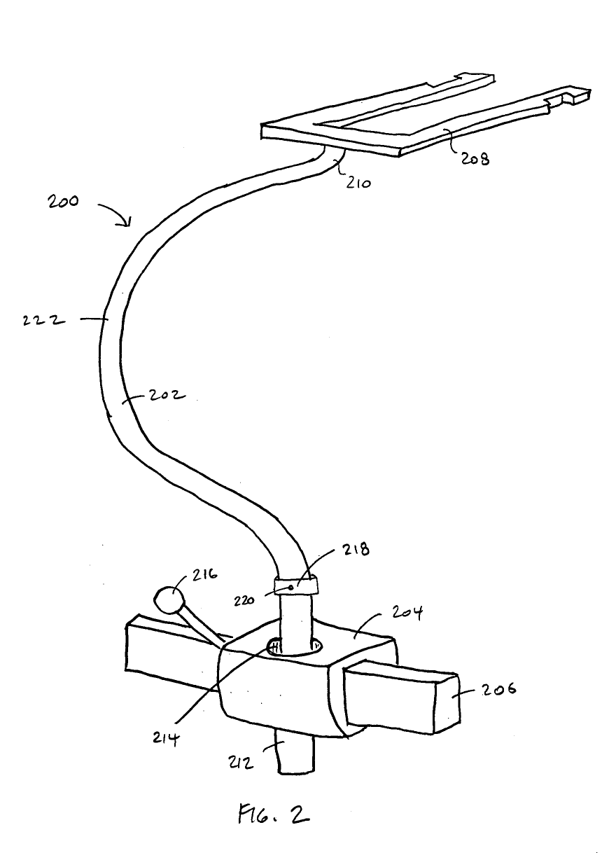

As shown in greater detail in FIG. 3, the platform mating device 208 may comprise a pair of legs, a first leg 224 and a second leg 226, extending from a base portion 228 to form an elongate U-shape. The legs 224, 226 and the base portion 228 are preferably formed into square sections, as shown in FIG. 3. At the distal ends 230, 232 of each leg 224, 226, away from the base portion 228, notches 234, 236 may be formed to accommodate a locking mechanism provided on the surgical platform 100, to attach the surgical platform to the post 202.

FIG. 4 depicts a cross-sectional view of the surgical platform 100 of FIG. 1 taken along section line A-A. In this view, both the first shaped passage 116 and the second shaped passage 118 can clearly be seen extending from the underside 124 of surgical platform 100. It will be appreciated that in order to install the surgical platform 100 upon the platform mating device 208, the first leg 224 and second leg 226 of the platform mating device may be slid into the apertures 126, 128 of the fist and second shaped passages 116, 118, respectively.

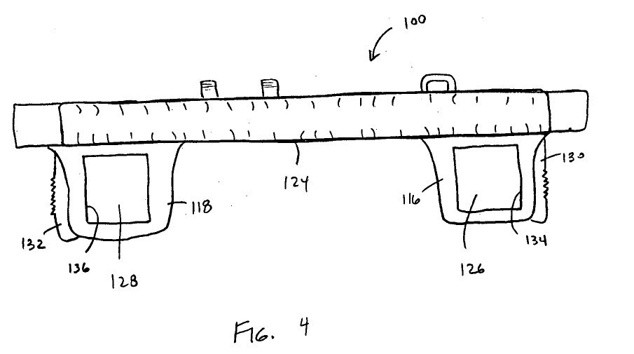

The shaped passages 116, 118 may include locking mechanisms such as the sliders 130, 132 shown. When the surgical platform 100 is pushed entirely upon the platform mating device 208, the notches 234, 236 of the first and second legs 224, 226 preferably align with the sliders 130, 132. In a first position, shown in FIG. 4, the sliders 130, 132 align with an interior wall of the shaped passages 116, 118 such that the legs 224, 226 may pass thereby. In a second position, shown in FIG. 5, the sliders 130, 132 may extend beyond the interior walls 134, 136 such that portions of the sliders extend into the apertures 126, 128, respectively. It will be appreciated that, if properly aligned, such movement will force the sliders 130, 132 into the notches 234, 236, to prevent relative movement between the platform mating device 208 and the surgical platform 100.

In use, the post 202 and mounting mechanism 204 are typically situated on the side of the surgical table opposite to the primary surgeon, such that the surgeon has access to the surgical platform 100 but is not inhibited by the post. Any adjustments that need to be made to the height of the platform 100 may be made by a surgical assistant from the side of the surgical table where the post is connected to the table.

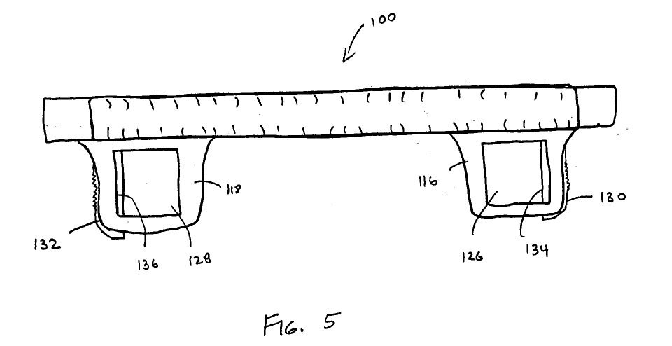

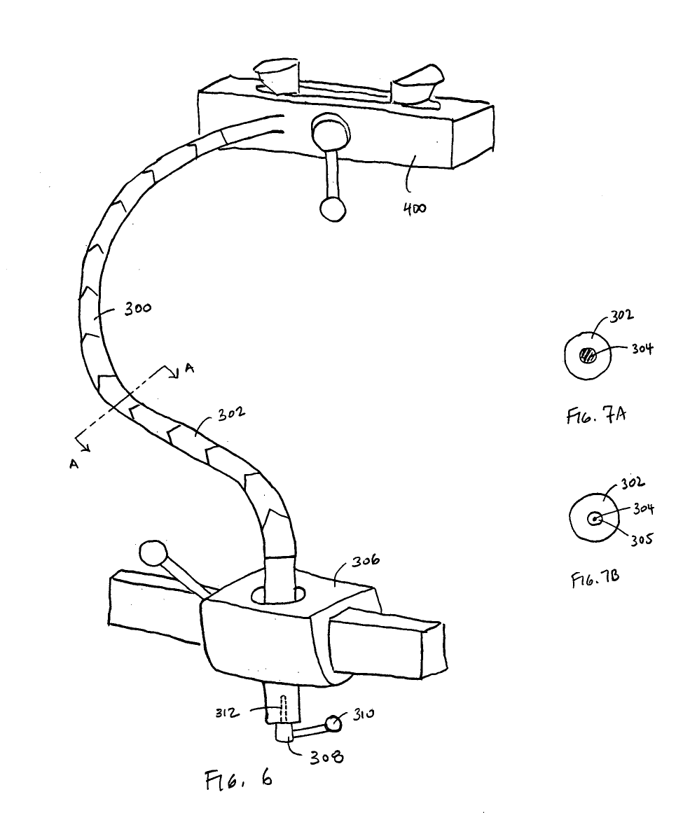

In accordance with another embodiment of the present invention, the post itself may be adjustable. An exemplary adjustable post is shown in FIG. 6. As shown, the post 300 may include a series of links 302 arranged in tandem. The links may be connected internally by a connecting device, such as a wire 304, shown in various conditions in FIGS. 7A and 7B, cross-sectional views of portions of the post 300 indicated by section A-A. The wire 304 may be relatively malleable yet firm, or completely flexible and loose. In the case of a firm wire 304, the wire may be bent internally within the post 300 such that movement of the links 302 permit the post to be deformed, where such deformation is held fairly stiff by the wire 304. It will be appreciated that certain metal wires, such as copper wires, may serve this purpose excellently. In this case, no tightening mechanism is required, as the wire 304 serves to support the post 300. The wire 304 is preferably sized in accordance with the load that it is intended to support, in that the links typically carry only a minimal load when a firm wire 304 is utilized. The wire 304 also will therefore preferably fill virtually the entire interior space of the link 302, as shown in FIG. 7A, leaving only a relatively small annular space.

Alternatively, the wire 304 may be completely flexible within the post 300. The flexible wire 304 may be selectively tightened or loosened by either one of a mounting mechanism 306, which may also operate to lock the height of the post 300, or a post tension device 308 adapted solely for this purpose. In this case, the wire 304 will preferably be thin, and not fill the interior space 305 of the link 302.

Using the post tension device 308 as an example, the post tension device may comprise a handle 310 which may be rotated around the longitudinal axis of the post 300, so as to rotate a spool 312 within the post. For clarity, the spool 312 is shown in FIG. 6 in phantom, although it is to be understood that the spool is preferably within the post 300. The spool 310 may cooperate with the wire 304 such that rotation of the spool 312 serves to wind the wire about the spool. The spool 312 may be unwound, the post 300 placed in a desired position, and the spool wound to tighten the wire 304 and bring each of the links 302 of the post into a compressive state against the adjacent link. The wire 304 may therefore be firmly affixed to the second end 311 of the post 300, opposite the mounting mechanism 306.

The links 302 may include surface irregularities on their ends, such as grooves, to increase the frictional interaction between adjacent links, thus helping to prevent links from slipping relative to one another as the wire 304 is tightened. In this case, the links 302 carry the weight of the supported devices.

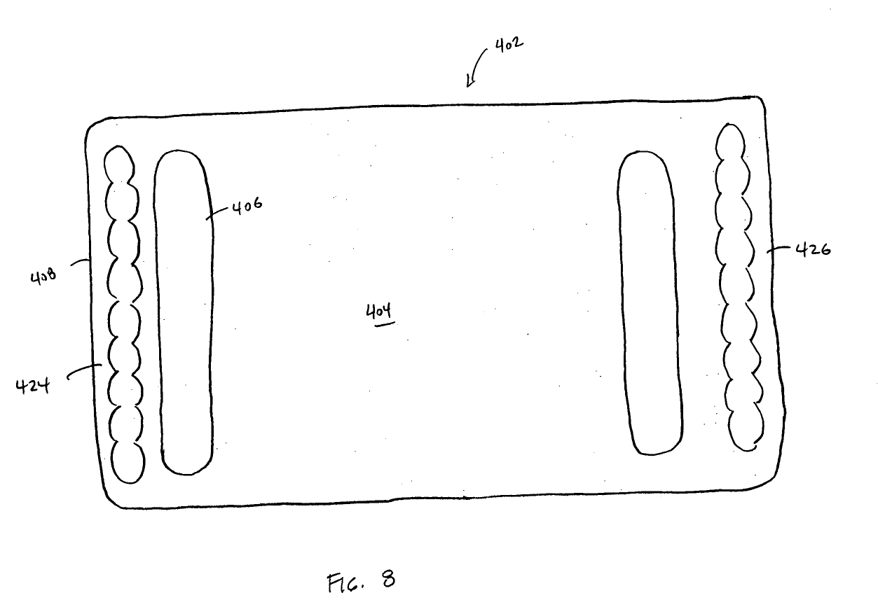

Also shown in FIG. 6 is a platform mounting mechanism in accordance with further aspects of the present invention attached to the second end 311 of the post 300. The platform mounting mechanism 400 shown in FIG. 6 is adapted to support a platform of the type shown in FIG. 8. As shown in FIG. 8, a plan view of the underside of a platform 402, a platform may comprise an underside 404 having at least one recessed channel 406 extending into the underside. The channel is preferably rectangular, and may be located in any position on the underside 404 of the platform. Preferably, the channel is along a side 408 of the platform 402, as shown in FIG. 8.

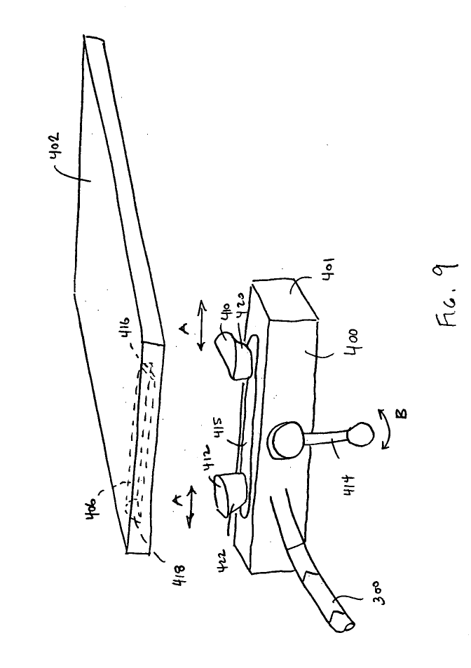

As shown in FIG. 9, the mounting mechanism 400 may be adapted to mount the platform 402 upon post 300. In this regard, the mounting mechanism 400 may include a pair of supports, consisting of a first support 410 and a second support 412, extending from a base 401. The supports 410, 412 may be arranged such that a crank 414 may move the supports together or away from each other within a channel 415, depending on the rotation of the crank. Mechanisms for such movement are well known in the art, and include geared screws and the like.

It will be therefore appreciated that the supports 410, 412 may be brought into proximity to each other, for example by counter-clockwise rotation of the crank 414. The platform 402 may then be placed over the supports 410, 412, such that the supports are within the channel 406. Once so aligned, rotation of the crank 414 in the opposite direction, for example in the clockwise direction, may serve to separate the supports 410, 412 such that the supports abut end portions 416, 418 of the channel 406. This abutment locks the platform 402 upon the mounting mechanism 400. Preferably the tolerances between them are small, so that little play between the two components is available.

The supports 410, 412 and channel 406 may include corresponding features to ensure that the platform 402 remains locked in position. For example, as shown in FIG. 9, the supports may include tapered portions 420, 422 which may butt up against similarly tapered portions of the channel 406, at the channel's ends 416, 418. Other features such as surface irregularities or male/female components may be utilized, the surface irregularities to increase the friction coefficient between the channel 406 and the supports 410, 412 and the male/female components to more positively lock the elements.

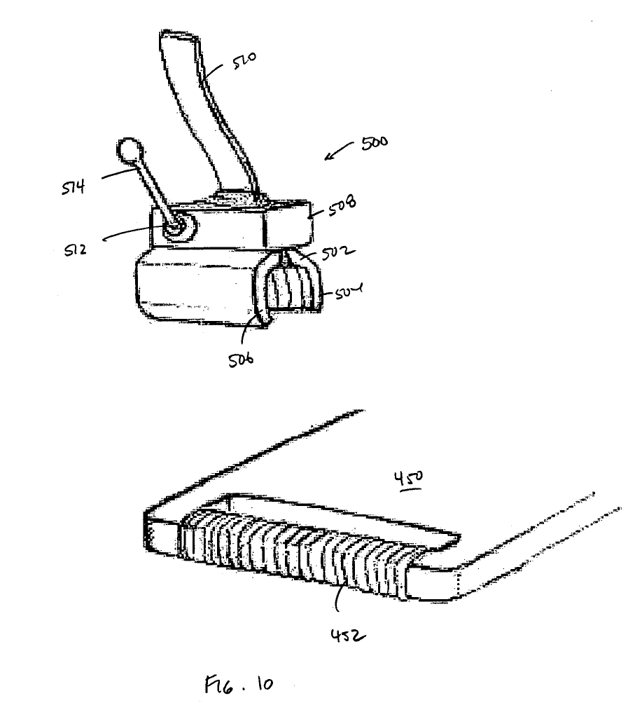

Referring back to FIG. 8, it is shown that a platform 402 may include handles 424, 426. Other types of platform handles are shown in FIG. 10, in accordance with other embodiments of the present invention. FIG. 10 depicts a portion of a platform 450, comprising a handle 452. The handle 452 may be utilized in the traditional sense to help a user carry the platform 450, but may also be used for other purposes.

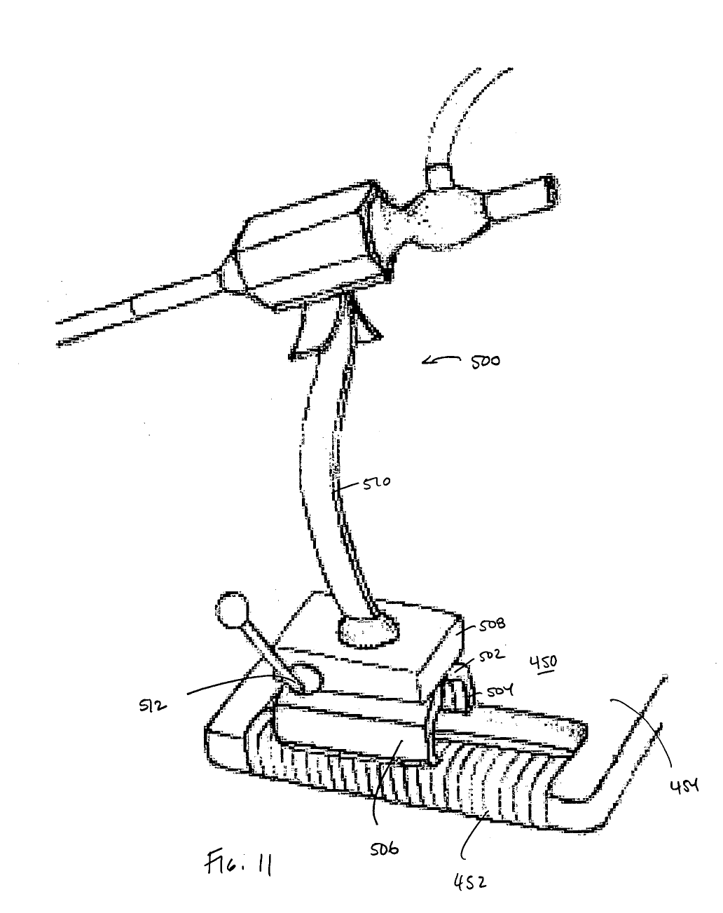

One such other purpose is to support accessories, such as the base portion of an accessory 500 shown in FIG. 10. Various types of accessories have been disclosed in the related applications, incorporated herein by reference. Any such accessory may be modified for this use and employed. For example, a camera mount accessory 500, as shown in FIG. 11, may be utilized.

Referring back to FIG. 10, it is shown that an accessory 500 may comprise a jaw mechanism 502 having a first jaw 504 and a second jaw 506. The jaw mechanism 502 may extend from a base portion 508 of the accessory 500. Extending from the base portion 508 in an opposite direction from the jaw mechanism may be a post 510. The post 510 may be a fixed post, or may incorporate any of the adjustment features detailed above, among others. The accessory 500 may also include a jaw manipulation component 512 operative by a handle 514. The jaw manipulation component may be utilized, such as by rotation of the handle, to move the first jaw 504 and the second jaw 506 relative to each other, such that the jaw mechanism 502 may close to grasp objects or open to release them.

One such object suited for grasping is the handle 452 of the platform 450, as shown in FIG. 11. In this regard, the handle 452 may be utilized to support accessories in a manner not previously realized. In order to accomplish such mounting, the jaw mechanism 502 may be opened, such that the first jaw 504, for example, may be placed between the handle 452 and the main portion 454 of the platform 450, with the second jaw 506 outside of the handle. The jaws 504, 506 may then be squeezed together by operation of the jaw manipulation component 512 and the jaw mechanism 502 to squeeze the handle 452. Once a sufficient force is exerted, it will be appreciated that the accessory 500 will be self-supportive upon the handle. The surgeon may then use the accessory 500 as needed in the surgical procedure. Preferably, the platform 450 is mounted to the surgical table using one of the novel mounting methods detailed above. Additionally, it is preferred that the jaws 504, 506 have a geometry which is shaped in correspondence with the handle 452. However, it need not be, as the jaws 504, 506 may include internal padding which allow the jaws to conform to a variety of shapes.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An assembly for supporting a surgical platform, said assembly comprising:
an attachment mechanism for securing said assembly to a surgical table;
a post extending from said attachment mechanism, said post having a first end at said attachment mechanism and a distal second end;
a platform mating device engaged with said second end of said post at a base portion, said platform mating device comprising at least one leg extending from the base portion, said at least one leg adapted to be mated with a surgical platform to support the surgical platform.

2. The assembly of claim 1, wherein said at least one leg further comprises a notch adapted to be mated with a locking member of a surgical platform, wherein the locking member of the surgical platform may enter the notch to restrict relative movement between the surgical platform and the at least one leg.

3. The assembly of claim 1, wherein said at least one leg comprises two legs, at least one of said legs having a notch adapted to be mated with a locking member of a surgical platform, wherein the locking member of the surgical platform may enter the notch to restrict relative movement between the surgical platform and the two legs.

4. The assembly of claim 1, wherein said first end of said post is relatively straight and is adapted to be adjustably mated with said attachment mechanism.

5. The assembly of claim 1, wherein said post comprises a series of links arranged in tandem.

6. The assembly of claim 5, wherein said post further comprises an internal wire, said wire serving to at least partially support said post.

7. The assembly of claim 6, wherein said links include aligned apertures therethrough, said wire nearly filling the entire apertures.

8. The assembly of claim 6, wherein said wire is malleable such that movement of said post by a user bends said wire into a fixed position at least partially supporting said post.

9. The assembly of claim 5, wherein said post further comprises an internal wire, said wire having a loose first condition and a taught second condition, said post being malleable when said wire is in said first condition and fixed when said wire is in said second condition.

10. The assembly of claim 9, wherein said post further comprises a post tensioning mechanism, said post tensioning mechanism adapted to selectively tighten or looses said wire within said post.

11. The assembly of claim 1, wherein said post is curved.

12. A post for mounting a surgical apparatus to a surface, said post comprising:
a first end and a second end, said first end operatively engaged with the surface and said second end operatively engaged with the apparatus;
a middle portion between said first end and said second end, said middle portion having at least one condition in which said middle portion is non-linear.

13. The post of claim 12, wherein said post comprises a series of links arranged in tandem.

14. The post of claim 13, wherein each of said links includes aligned apertures, said post further comprising a wire running along the length of said post within said apertures.

15. The post of claim 14, wherein said wire leaves an annular space between said wire and said links.

16. The post of claim 14, wherein said wire is malleable such that movement of said post by a user bends said wire into a fixed position at least partially supporting said post.

17. The post of claim 16, wherein said wire at least partially supports said post.

18. The post of claim 14, wherein said wire has a first condition in which said wire is loose and a second condition in which said wire is tight, said post being flexible in said first condition and rigid in said second condition.

19. The post of claim 18, wherein said post further comprises a post tensioning device operative to selectively tighten or loosen said wire within said post.

20. The post of claim 18, wherein said links comprise features on their end surfaces to increase friction between each of said links.

21. The post of claim 18, wherein said links include grooves on their end surfaces.

22. An assembly for supporting a surgical platform, said assembly comprising:
an attachment mechanism for securing said assembly to a surface;
a post extending from said attachment mechanism,
a platform mating mechanism engaged with said post at a base portion opposite said attachment mechanism, said platform mating mechanism comprising a pair of opposed supports operative to selectively move toward each other or away from each other, said supports adapted to be mated with a surgical platform to support the surgical platform.

23. The assembly of claim 21, wherein said platform mating mechanism further comprises a base having a channel therein, said supports moveable within said channel.

24. The assembly of claim 21, wherein said supports include tapered outside edges.

25. A surgical platform system, said system comprising:
a surgical platform, said surgical platform comprising an underside having a recessed channel therein;
a platform support mechanism, said platform support mechanism comprising an attachment mechanism for attachment of said platform support mechanism to a surface, a platform mating mechanism, and a post extending between said attachment mechanism and said platform mating mechanism, said platform mating mechanism comprising a pair of adjustable supports, said supports adapted to be placed within said channel.

26. The surgical platform system of claim 25, wherein said supports have a first condition in which said supports are spaced apart from each other and a second condition in which said supports are moved together, said supports adapted to be matingly engaged with said recessed channel when said supports are in said first condition.

27. The surgical platform system of claim 25, wherein said mating engagement is by way of frictional forces between said supports and said recessed channel.

28. The surgical platform system of claim 25, wherein said supports are tapered.

29. The surgical platform system of claim 28, wherein said recessed channel is correspondingly tapered such that the tapered supports and the tapered recessed channel affix said surgical platform to said platform by more than friction alone.

30. A surgical platform comprising a main portion and at least one shaped passage below said main portion, said shaped passage adapted to permit entry of a platform mating device for supporting said platform.

31. The surgical platform of claim 30, wherein said at least one shaped passage comprises a rectangular cross-section.

32. The surgical platform of claim 30, further comprising a locking device associated with said shaped passage, said locking device having a first position in which said locking device at least partially enters the shaped passage and a second position in which said locking device is not within said shaped passage.

33. The surgical platform of claim 32, wherein said shaped passage includes a longitudinal centerline and said locking device enters said shaped passage normal to said longitudinal centerline.

34. A device for attaching a surgical accessory to a surgical platform having a handle and a main portion, said device comprising a jaw mechanism, said jaw mechanism comprising a first jaw adapted to fit between the handle of the surgical platform and the main section of the surgical platform and a second jaw adapted to be outside the handle such that the jaw may be closed to squeeze the handle of the surgical platform to support the device.

35. The device of claim 34, further comprising a post, said post adapted to support a surgical accessory.

36. The device of claim 34, further comprising a jaw manipulation component, wherein said jaw manipulation component is adapted to move said jaw from an open position to a closed position to selectively squeeze the handle.

37. The device of claim 34, wherein said first jaw and said second jaw are curved.

38. The device of claim 34, wherein said first jaw and said second jaw have a geometry corresponding to the shape of the handle.
